(19) United States
(12) Patent Application Publication
(10) Pub. No.: US 2015/0132273 A1
(43) Pub. Date: May 14, 2015

(54) Nutritional Compositions and Methods for Treating Cognitive Impairment

(71) Applicant: Rhett Sean Daniels, Ft. Myers, FL (US)

(72) Inventor: Rhett Sean Daniels, Ft. Myers, FL (US)

(21) Appl. No.: 14/144,012

(22) Filed: Dec. 30, 2013

Related U.S. Application Data
(60) Provisional application No. 61/902,199, filed on Nov. 9, 2013.

(51) Int. Cl. A61K 38/53; A61K 31/714; A61K 33/06; A61K 31/198; A61K 31/202; A61K 31/661; A61K 31/59; A61K 31/51; A61K 31/525; A61K 31/455; A61K 31/375; A61K 31/191; A61K 38/48; A61K 31/4525; A61K 31/7076; A23L 1/30; A61K 31/519

(52) U.S. Cl. CPC A61K 38/53; A61K 31/519; A61K 31/714; A61K 33/06; A61K 31/198; A61K 31/202; A61K 31/661; A61K 31/59; A61K 31/51; A61K 31/525; A61K 31/455; A61K 31/375; A61K 31/191; A61K 38/4873; A61K 31/4525; A61K 31/7076; A23L 1/30
USPC 424/94.2

(57) ABSTRACT

A nutritional supplement that contains folinic acid, folic acid, USP, methylfolic acid, cobalamin, magnesium, docosahexaenoic acid, phosphatidylcholine-docosahexaenoic acid, phosphatidylethanolamine-docosahexaenoic acid.

A nutritional supplement is disclosed for the treatment of cognitive disorders, attention deficit disorder, oxidative stress, depression, memory loss, anxiety, and apathy.

### CROSS-REFERENCE TO RELATED APPLICATION

This application is a nonprovisional which claims priority to U.S. Provisional Patent Application Ser. No. 61/902,199 filed Nov. 9, 2013, which is hereby incorporated by reference in its entirety.

### FIELD

The present disclosure generally relates to compositions made of folates, B Vitamins, and other vitamins and minerals for treating a number of cognitive impairments.

### BACKGROUND

Folic acid and more particularly reduced folates are important vitamins that play many roles in providing mammals with proper nutrition. Many people have cognitive impairments that can be alleviated, treated, and sometimes completely reverse with a proper balance of nutrition through vitamin and mineral supplementation.

### SUMMARY

A nutritional supplement that contains folinic acid, folic acid, USP, methylfolic acid, cobalamin, magnesium, docosahexaenoic acid, phosphatidylcholine-docosahexaenoic acid, phosphatidylethanolamine-docosahexaenoic acid.

A nutritional supplement that contains folinic acid, folic acid, USP, methylfolic acid, cobalamin, magnesium, docosahexaenoic acid, phosphatidylcholine-docosahexaenoic acid, phosphatidylethanolamine-docosahexaenoic acid, Vitamin D₃, Vitamin B₁, Vitamin B₂, Vitamin B₅, Vitamin B₃, Vitamin C, Threonate, N-acetyl-L-cysteine amide, enzyme, intrinsic factor, and piperine.

A nutritional supplement for the treatment of cognitive disorders, attention deficit disorder, oxidative stress, anxiety, and apathy that contains folinic acid, folic acid, USP, methylfolic acid, cobalamin, magnesium, docosahexaenoic acid, phosphatidylcholine-docosahexaenoic acid, phosphatidylethanolamine-docosahexaenoic acid.

### DETAILED DESCRIPTION

The present disclosure will now be described more fully hereinafter, in which some, but not all embodiments of the disclosure are shown. Indeed this disclosure may be embodied in many different forms and should not be construed as limited to the embodiments set forth hereinafter; rather, these embodiments are provided so that this disclosure will satisfy applicable legal requirements

This disclosure describes embodiments for orally administered prescription folate containing products for the clinical dietary management of mild to moderate cognitive impairment disorders related to suboptimal folate levels. Many of these conditions are associated with metabolic imbalances in transformylation and/or methylation biochemistry with particular emphasis on dopamine stimulated phospholipid methylation—a unique membrane delimited signaling process mediated by the D4 dopamine receptor that promotes neuronal synchronization and attention. It may also play a role in regulating oxidative stress, mitochondrial dysfunction, inflammation, and proteasome impairment. The protective effect of providing three different forms of folate through folic acid, folinic acid, and L-methylfolate provides for improved nutrition treating a number of conditions is described herein. These embodiments provide supplying an array of folate derivatives in combination with folate coenzymes, cofactors and co-metabolites that decrease the risk of bioavailability interference such as might occur with inborn or environmental folate malabsorption.

The embodiments of the present disclosure allow the folate substrate, THF, to be utilized in transformylation and/or methylation biochemistry. The embodiments include suppliments that may be taken by women of childbearing age, but is not indicated for pregnancy or lactation. The nutritional supplements may also be taken by a number of individuals having different cognitive problems from depression, memory loss, apathy, and attention deficit disorder (ADD).

Folic acid has received considerable attention as an essential vitamin. It is widely used in prenatal vitamins to reduce the risk of neural tube defects, but it also holds a prominent position in a multitude of other non-prenatal products. Indeed, as additional research emerges definitively linking homocysteine to varied disease states, it is anticipated that the importance of supplemental folate will be further embraced by the healthcare industry. Folic acid may be provided in differing amounts ranging from 0.2 mg to 3 mg.

Vitamin B₁₂ is essential for appropriate folic acid metabolism, which is why including Vitamin B₁₂ in a nutritional supplement with folic acid would be beneficial. When a person is deficient in Vitamin B₁₂, it may be noted by megaloblastic anemia. Vitamin B₁₂ also plays a role in maintaining cellular integrity of the central nervous system. Therefore, while supplementation of folic acid may cure hematologic symptoms (anemia) of B₁₂ deficiency, it will leave a fetus vulnerable to central nervous system damage. Vitamin B₁₂ is found exclusively in animal tissues hence during pregnancy a vegan woman is at risk for B₁₂ deficiency. Vitamin B₁₂, may be provided as cyanocobalamin to mitigate any deficiency of this essential vitamin. Vitamin B₁₂ may be provided in differing amounts ranging from 0.5 mg to 10 mg.

Vitamin B₆ is a water-soluble vitamin and is part of the vitamin B complex group. Vitamin B₆ is a cofactor in many reactions of amino acid metabolism, including transamination, deamination, and decarboxylation. The primary role of vitamin B₆ is to act as a coenzyme to many other enzymes in the body that are involved predominantly in metabolism. This role is performed by the active form, pyridoxal phosphate. Pyridoxal phosphate-dependent enzymes play a role in the biosynthesis of five important neurotransmitters: serotonin, dopamine, epinephrine, norepinephrine, and gamma-aminobutyric acid (GABA). Vitamin B₆ may be provided in differing amounts ranging from 0.2 mg to 1.5 mg.

Magnesium may be supplemented to prevent pre-eclampsia, preterm rupture of the membranes, and preterm births secondary to early labor. Women who deliver preterm are more likely to have lower plasma levels of this mineral. Magnesium may be provided in differing amounts ranging from 50 mg to 500 mg (elemental).

Iron is necessary for blood formation of the fetus and placenta. Iron depletion and iron deficiency anemia are the most common deficiency in pregnancy (90% of all anemias). Growth restriction, preterm delivery, and pre-eclampsia have been noted in women who have iron deficiency. Iron may be provided in differing amounts ranging from 5 mg to 50 mg.

Vitamin B₂ deficiency has been associated with fetal malformation of the bony tissue and membranous skeleton, which precedes the cartilageous and osseous skeletons. Proper nutrition of Vitamin B₂ is also important to prevent hyperemesis gravidarum and an increased incidence of growth restriction and preterm delivery in the fetus. Maternal deficiency of Vitamin B₂ is associated with stomatitis, glossitis and cheilosis. Vitamin B₂ may be provided in differing amounts ranging from 0.3 mg to 3 mg.

Ascorbic acid (Vitamin C) is essential for the formation of collagen and therefore is very important for both mother and fetus during pregnancy. The transport mechanism across the placenta is the same for that of glucose therefore Vitamin C supplementation is very important in those women having (or at risk for) diabetes. Vitamin C may be provided in differing amounts ranging from 5 mg to 50 mg.

Vitamin B₅/pantethine/coenzyme A: Pantothenic acid is an essential nutrient. Animals require pantothenic acid to synthesize coenzyme-A (CoA), as well as to synthesize and metabolize proteins, carbohydrates, and fats. Pantothenic acid participates in a wide array of key biological roles, it is essential to all forms of life. Vitamin B₅ may be provided in differing amounts ranging from 1 mg to 10 mg.

Omega-3 fatty acids may be found in marine fats. They have been shown to be important in the prevention of pre-eclampsia, preterm delivery, and early rupture of the membranes. Enhanced cognitive function and improved visual acuity in babies born to mothers supplemented with docosahexaenoic acid (DHA) have also been noted. There has been a decrease in maternal postpartum depression when supplemented with DHA. Cold water fish are the highest dietary sources of DHA and it is also available in the eggs of chickens supplemented with micro-algae. DHA may be provided in differing amounts ranging from 30 mg to 150 mg.

Vitamin D regulates calcium homeostasis by maintaining equilibrium (along with parathormone) between calcium resorption and excretion. If Vitamin D levels are low, then the mother may lose significant calcium in her urine. If maternal calcium intake is low, then poor bone mineralization is likely to occur in infants. Vitamin D may be in the form of Vitamin D₃ or cholecalciferol. Vitamin D may be provided in differing amounts ranging from 20 IU to 600 IU.

Proper Zinc nutrition helps prevent congenital malformations as well as fetal losses. Since maternal plasma levels of zinc decrease during pregnancy, supplementation is important. In the fetus, deficiency of zinc may be involved with premature rupture of the membranes and a reduced ability to fight infection due to suppressed immunity.

Zinc availability affects brain glutathione. A deficit in zinc availability can increase cell oxidant production, affect the antioxidant defense system, and trigger oxidant-sensitive signals in neuronal cells. GSH levels can become low with zinc deficiency. γ-Glutamylcysteine synthetase (GCL), the first enzyme in the GSH synthetic pathway, may also be altered by zinc deficiency. The protein and mRNA levels of the GCL modifier and catalytic subunits may be lower in zinc deficient. The nuclear translocation of transcription factor nuclear factor (erythroid-derived 2)-like 2, which controls GCL transcription, may be impaired by zinc deficiency. Zinc may be provided in the amounts ranging from 4 mg to 20 mg.

Low zinc also exacerbates the effects of stress on the body and accelerates aging. Additionally, adequate zinc is necessary for optimal physical performance, energy levels, and body composition. Zinc affects protein synthesis and is required for proper function of red and white blood cells. It is highly concentrated in our bones, the pancreas, kidneys, liver, and retina.

Zinc is an excellent antioxidant. Antioxidants get rid of free radicals that cause damage to cells in the body by bonding with them and neutralizing them. Zinc is particularly good at countering the damaging effect of high iron. Zinc also targets free radicals that cause inflammation and is especially effective at detoxifying heavy metals from the brain.

The super antioxidant effects of zinc allow it to efficiently remove toxins from the body and keep them from building up in tissue and causing damage. The progression of neurodegeneration and Alzheimer's disease is accelerated by heavy metal buildup in the brain. Zinc can help get rid of those toxins, and it also helps maintain cellular homeostasis of brain cells.

Zinc plays a key role in neurotransmitter function and helps to maintain brain structure and health. It is necessary in the metabolism of melatonin, which regulates dopamine. Also, zinc is part of an enzyme that is necessary for the anabolism of fatty acids in the brain membrane. This is very important because a key part of supporting brain health and function is to ensure the membrane gets the nutrients it needs.

Reduced Vitamin B₉: The health benefits and even requirements of folic acid have been discussed previously. In addition to folic acid, reduced folates are also beneficial for some people's nutrition. Folic acid is the most oxidized state of Vitamin B₉. Reduced folates are more bioactive and are more readily available for the cells to use. A combination of folic acid with reduced folates has shown to have a synergistic effect.

Folate mechanism of action: Folate is essential for the production of certain coenzymes in many metabolic systems such as purine and pyrimidine synthesis. It is also essential in the synthesis and maintenance of nucleoprotein in erythropoiesis. It also promotes white blood cell (WBC) and platelet production in folate-deficiency anemia. Folate is associated with methylation and transformylation biochemistry.

Pharmacology: Folate—Folates are best known for reducing the incidence of fetal neural tube defects (NTDs). NTDs are congenital malformations produced by failure of the neural tube to form and close properly during embryonic development. During the first four weeks of pregnancy—when many women do not even realize that they have conceived, adequate maternal folate intake is essential to reduce the risk of NTDs. As the postnatal period approaches there is increased demand again for folate regardless of lactation status. Folate is involved in transformylation and methylation metabolism as well as—indirectly, succinylation metabolism (through the “methyl trap” hypothesis). Folate plays a central role in the formation of nucleic acid precursors, such as thymidylic acid and purine nucleotides, which are essential for nucleic acid synthesis and cell division. There's evidence that suggests the protective effect from folate supplements is much stronger than that for food folate. Other dietary ingredients are added to folate as cofactors, coenzymes and co-metabolites. Factors other than folate intakes may affect the magnitude of risk reduction or participate in a co-protective effect with folate.

The present forms of folate in the embodiments of this disclosure are:

Folic acid, which in one embodiment may be in the form of a controlled-release delivery method to optimize absorption of folic acid. In one embodiment, the controlled release form is achieved from citrated folic acid pellets. These citrated folic acid pellets are comprised of conventional folic acid encapsulated with citrates, in controlled-release pellets. These citrated folic acid pellets are a unique from conventional folic acid in that it: 1.) complies with USP requirements for folic acid dissolution. Some studies indicate that dissolution failure—that is, the failure of conventional folic acid supplements to meet USP requirements for dissolution—is a significant, concerning problem. 2.) includes buffers to adjust the pH in order to remain soluble in a high acid environment, such as the gastric environment. This is important because folic acid must remain soluble in the acidic environment of the stomach in order to be absorbed in the intestine. Studies show that solubility of conventional folic acid decreases with increased acidity. Folic acid is converted into functional, metabolically active coenzyme forms for use in the body (61 Fed. Reg. at 8759-60), and supplies the active folate substrate, THF (tetrahydrofolate). Folic acid may be provided in differing amounts ranging from 0.1 mg to 1.5 mg.

Folinic Acid—Folinic acid plays an essential role in transformylation reactions, and is also the immediate precursor of L-5-MTHF substrate for B₁₂-assisted methionine synthase which remethylates homocysteine to methionine. Folinic acid is a metabolically active folate, and does not require reduction by dihydrofolate reductase (DHFR). DHFR is an extremely slow enzyme with some individuals possessing lower than average activity. Studies of patients with cerebral folate deficiency have shown that oral folinic acid can bypass defective folate receptors and restore central nervous system folate levels. Low folate status is associated with impaired cognitive function. Folinic acid is converted into functional, metabolically active coenzyme forms for use in the body, and supplies the active folate substrate, THF (tetrahydrofolate). Folinic acid may be provided in differing amounts ranging from 0.5 mg to 10 mg.

Methylfolic acid—About 70% of food folate and cellular folate is comprised of L-methylfolate. It is the primary form of folate in circulation, and is also the form transported across membranes—particularly across the blood brain barrier—into peripheral tissues. In the cell, L-methylfolate is used in the remethylation of homocysteine to form methionine and tetrahydrofolate (THF). Elevated levels of homocysteine may induce oxidative stress, leading to blood-brain barrier (BBB) dysfunction. L-methylfolate is converted into functional, metabolically active coenzyme forms for use in the body, and supplies the active folate substrate, THF (tetrahydrofolate).). L-methylfolate may be provided in differing amounts ranging from 0.2 mg to 3 mg.

Pharmacology: Folate Coenzymes, Cofactors and Co-Metabolites—The following dietary ingredients are added to enhance the bioavailable potential of folate, and include:

Hydroxocobalamin—Hydroxocobalamin is a naturally occurring member of the vitamin B₁₂ family. Hydroxocobalamin exists in aqueous solution as an equilibrium mixture of the hydroxocobalamin and aquacobalamin isomers. Aquacobalamin is a precursor of the cobalamin metabolite glutathionylcobalamin (GSCbl, a precursor for the two coenzyme forms of vitamin B₁₂, adenosylcobalamin and methylcobalamin). Cobalamin is required for two important reactions: the conversion of methylmalonyl CoA to succinyl CoA, a Krebs cycle intermediate, and the conversion of homocysteine to methionine, a reaction in which the methyl group of methyltetrahydrofolate is donated to remethylate homocysteine. Cobalamin deficiency, even in the absence of hematologic signs, may lead to impaired cognitive performance in adolescents. Decreased cobalamin status is strongly associated with cognitive dysfunction in the elderly. Many factors contribute to the cobalamin deficiency including diet, gastrointestinal pathology, autoimmune disease and medications. Hydroxocobalamin may be provided in differing amounts ranging from 1 mg to 10 mg.

Magnesium—Many neurodegenerative conditions have been reported to be associated with decreased brain magnesium levels and increased oxidative stress. Magnesium is an obligatory cofactor in glutathione synthesis. As a result, magnesium deficiency may impair glutathione synthesis. Magnesium chelated with N-acetyl-L-cysteine provides magnesium as magnesium-N-acetyl-L-cysteine mineral amino acid derivative chelate and may increase both brain magnesium and glutathione levels. Magnesium concentrations have been shown to affect serotonin receptors, nitric oxide synthesis and release and NMDA receptors.

Essential Fatty Acids—Docosahexaenoic acid (DHA) is an omega-3 fatty acid. DHA is the predominant fatty acid in the brain and is found primarily conjugated to phospholipids. The PC-DHA and PE-DHA that may be incorporated in the embodiments of this disclosure are body-ready, natural phospholipid omega-3 fatty acids. Specific nutrient deficiencies have been described in children with Attention Deficit Disorder including essential fatty acids. Higher levels of DHA improved cognitive performance in middle-aged adults. PC-DHAe as phosphatidylcholine-docosahexaenoic acid may be provided in differing amounts ranging from 400 mcg to 1 mg. PE-DHAe as phosphatidylethanolamine-docosahexaenoic may be provided in differing amounts ranging from 100 mcg to 600 mcg.

Vitamin D—Cholecalciferol is a form of vitamin D, also called vitamin D₃. Vitamin D receptors are widespread in brain tissue, and vitamin D's biologically active form has shown neuroprotective effects. Associations have been noted between low vitamin D status and Alzheimer's disease and dementia in both Europe and the US. Vitamin D deficiency is seen frequently among the elderly. Vitamin D may be provided in differing amounts ranging from 20 IU to 600 IU.

B Vitamins—An association between inadequate vitamin B status and cognitive decline has been shown in studies. Mild subclinical vitamin deficiencies of niacin, thiamine, and riboflavin, are not uncommon in individuals with cognitive impairment. Emerging evidence suggests that thiamine deficiency produces alterations in brain function and structural damage that closely models a number of diseases in which neurodegeneration is a characteristic feature, including AD and PD; thus Thiamine may have a role in AD and PD. Niacin may be provided in differing amounts ranging from 300 mcg to 3 mg. Riboflavin may be provided in differing amounts ranging from 300 mcg to 3 mg. Thiamine may be provided in differing amounts ranging from 300 mcg to 3 mg.

Coenzyme A—Coenzyme A sometimes known as Vitamin B₅ (from pantethine) is a precursor to acetyl-coenzyme A which is involved in synthesis and oxidation of fats. Coenzyme A may be provided in differing amounts ranging from 1 mg to 10 mg.

Vitamin C—Vitamin C assists with folate metabolism. Studies also show that vitamin C may protect against cognitive impairment in some individuals. It also helps the absorption and bioavailability of folates.

Threonate—L-threonate magnesium is a highly absorbable form of threonic acid that is able to increase brain magnesium levels. Elevation of brain magnesium may prevent cognitive deficits. Magnesium deficiency can arise from diminished intake of magnesium, such as observed in malnutrition and starvation. Another cause of magnesium deficiency is excessive magnesium excretion, such as seen in uncontrolled diabetes, severe diarrheal states, malabsorption, and diuretic therapy. Magnesium deficiency may affect vitamin D absorption.

NACA—N-acetyl-1-cysteine amide is the amide form of acetylcysteine—an amino acid derivative that has been shown to cross the blood-brain barrier. Acetylcysteine is a precursor to the antioxidant glutathione—the most prevalent circulating antioxidant. Oxidative stress has been implicated in cognitive disorders, such as mild cognitive impairment NACA may be provided in the amounts ranging from 2 mg to 25 mg.

Enzyme—Papaya proteinase I is a protein-cleaving enzyme derived from papaya. Papaya proteinase I may free cobalamin from unabsorbable cobalamin complexes (cobalamin-R-proteins) formed in situations of pancreatic insufficiency. Exocrine pancreatic dysfunction is associated with impaired ileal absorption of cobalamin. Papaya proteinase I may be provided in the amounts ranging from 5 mg to 45 mg.

Intrinsic Factor—Intrinsic factor (IF) is a protein secreted by gastric parietal cells that facilitates absorption of cobalamin in the ileum. Once cobalamin is released into the intestinal lumen, it is taken up by intrinsic factor. The cobalamin-intrinsic factor complex is then absorbed by cells in the ileum, where the cobalamin is released and transported to the blood stream. Atrophic gastritis, reported to occur with a high prevalence in elderly patients, may result in decreased intrinsic factor secretion and consequently, impaired cobalamin absorption. Intrinsic factor may be provided in the amounts ranging from 1 mg to 30 mg.

Bioavailability enhancer—Piperine is an alkaloid found naturally in plants. Piperine may have bioavailability-enhancing activity for some nutritional substances and for some drugs. It has putative anti-inflammatory activity and may have activity in promoting digestive processes as well as may inhibit lipid peroxidation. The exact mechanism of action is unknown. Piperine has been shown to stimulate the secretion of digestive enzymes pancreatic amylase, trypsin, chymotrypsin, and lipase in rats.

Dosage and administration: One embodiment provides one tablet to be taken daily or as directed under medical supervision to achieve a satisfactory folate-maintenance level. In one embodiment, the nutritional supplement is a chewable tablet, in another body a tablet or capsule. In one embodiment, the supplement is coated and may be swallowed. This disclosure may also be administered by manually crushing a tablet, and then adding the contents to a thick juice or soft food—such as applesauce or yogurt.

In one embodiment, this supplement is not a drug, but may be used as monotherapy (“rescue” therapy) or adjunctive therapy as determined by a licensed medical practitioner. The adjunctive use of the embodiments of this disclosure enable medical practitioners to combine therapeutic modalities (dietary management and drug therapy). In patients with suboptimal folate levels—and as determined by a licensed medical practitioner. The embodiments of this disclosure may be administered as rescue or adjunctive folate-therapy to provide a protective effect in reducing the risk of secondary/endpoints and/or disease-states of a vascular nature such as may be found with generalized cognitive decline, Vascular Dementia or Alzheimer's. In one embodiment, a supplement of this disclosure may be administered as rescue or adjunctive folate-therapy to provide a protective effect in reducing the risk of secondary/endpoints and/or disease-states of a dopamine-methylation metabolic imbalance as may be found with Parkinson's, Huntington's, Fibromyalgia, Chronic Fatigue Syndrome and/or attention-deficit/hyperactivity disorder (ADHD) patients. The embodiments of this disclosure do not contain vitamin B₆, and therefore has less risk of excessive dopamine production and antagonizing/agonizing dopamine-targeting drug therapies such as LevoDopa/CarbiDopa PK therapy. These embodiments also do not contain iron, and therefore has less risk of iron overload—a concern for elderly populations with or without neurodegenerative disorders⁴².

### Example One

### Ingredients

Each Multiphasic-Chewable, Coated-Orange Tablet contains the following 5 mg of active folic acid/vitamin B₉ moiety from DeltaFolate™:

| | | |
|---|---|---|
| Folinic Acid [from formyltetrahydrofolic acid]. | 4 | mg |
| Folic Acid, USP as CitraFolic ™ [from controlled-release | 0.3 | mg |
| pteroylmonoglutamic acid] | | |
| Methylfolic Acid [from methylfolate magnesium] | 0.7 | mg |
| Cobalamin [from hydroxocobalamin]. | 2 | mg |
| Magnesium as magnesium n-acetyl-l-cysteined amino acid | 5 | mg |
| derivative chelate | | |
| DHA as docosahexaenoic acid | 80 | mg |
| PC-DHAe as phosphatidylcholine-docosahexaenoic acid | 700 | mcg |
| PE-DHAe as phosphatidylethanolamine-docosahexaenoic | 300 | mcg |
| acid | | |
| Vitamin D₃ [from cholecalciferol] | 100 | IU |
| Vitamin B₁ [from thiamine]. | 1 | mg |
| Vitamin B₂ [from riboflavin]. | 1 | mg |
| Coenzyme A/Vitamin B₅ [from pantethine]. | 5 | mg |
| Vitamin B₃ [from niacin] | 1 | mg |
| Vitamin C [from l-ascorbic acid]. | 25 | mg |
| Threonate [from l-threonate magnesium] | 5 | mg |
| Amino acid derivative [from NACA as n-acetyl-l-cysteine | 10 | mg |
| amide] | | |
| Enzyme [from papaya proteinase I] | 20 | mg |
| IF [from intrinsic factor] | 5 | mg |
| Bioavailability Enhancerf [from piperine] | 250 | mcg |

Excipients:

Citric acid, fumed silica, magnesium stearate, microcrystalline cellulose, natural orange color coating (beta carotene, hydroxypropyl cellulose, hydroxypropyl methyl cellulose, natural orange flavor, polyvinyl alcohol, polyethylene glycol, riboflavin, stevia, talc, titanium dioxide), natural orange flavor, Saccharomyces boulardii (trace amounts), silicon dioxide, stearic acid, stevia, sucrose, tapioca starch . . . [and other ancillary ingredients as needed to ensure product stability] . . . . Other excipients known in the art may be used as alternatives.

### Example Two

### Description

This disclosure is an orally-administered prescription folate-containing product for the clinical dietary management of mild-to-moderate cognitive impairment disorders related to suboptimal folate levels associated with metabolic imbalances in transformylation and/or methylation biochemistry.

Ingredients:

Each round, Multiphasic-Chewable, Coated-Orange Tablet contains the following 4.8 mg of active vitamin B₉ moiety from 8 mg DeltaFolate™ provided as:

| | | |
|---|---|---|
| Formyltetrahydrofolic acid [equivalent to 4 mg folinic | 5 | mg |
| moiety] | | |
| Controlled-release citrated-pteroylmonoglutamic acid, USP | 2 | mg |
| [equiv. to 0.3 mg folic moiety] | | |
| Methylfolate magnesium [equivalent to 0.5 mg methylfolic | 1 | mg |
| moiety]. | | |
| Magnesium n-acetyl-l-cysteine. | 5 | mg |
| NACA as n-acetyl-l-cysteine amide | 10 | mg |
| PC-DHA as phosphatidylcholine-docosahexaenoic acid | 700 | mcg |
| PE-DHA as phosphatidylethanolamine-docosahexaenoic | 300 | mcg |
| acid. | | |

In addition to above, each chewable tablet also contains vitamin D, B vitamins, vitamin C and other dietary ingredients.

### Example Three

### Ingredients

Each Multiphasic-Chewable, Coated-Orange Tablet contains the following 5 mg of active folic acid/vitamin B₉ moiety from DeltaFolate™:

| | |
|---|---|
| Folinic Acid [from formyltetrahydrofolic | From 1 mg to 10 mg |
| acid]. | |
| Folic Acid, USP as CitraFolic ™ [from | From 0.1 mg to 1.3 mg |
| controlled-release pteroylmonoglutamic | |
| acid].. | |
| Methylfolic Acid [from methylfolate | From 0.2 mg to 2 mg |
| magnesium] | |
| Cobalamin [from hydroxocobalamin]. | from 1 mg to 10 mg |
| Magnesium as magnesium n-acetyl-l- | from 1 mg to 10 mg |
| cysteined amino acid derivative chelate | |
| DHA as docosahexaenoic acid | from 30 mg to 150 mg |
| PC-DHAe as phosphatidylcholine- | from 400 mcg to 1 mg |
| docosahexaenoic acid | |
| PE-DHAe as phosphatidylethanolamine- | from 100 mcg to 600 mcg |
| docosahexaenoic acid | |
| Vitamin D₃ [from cholecalciferol] | from 20 IU to 600 IU |
| Vitamin B₁ [from thiamine]. | from 300 mcg to 3 mg |
| Vitamin B₂ [from riboflavin]. | from 300 mcg to 3 mg |
| Coenzyme A/Vitamin B₅ [from pantethine]. | from 1 mg to 10 mg |
| Vitamin B₃ [from niacin] | from 300 mcg to 3 mg |
| Vitamin C [from l-ascorbic acid]. | from 5 mg to 50 mg |
| Threonate [from l-threonate magnesium] | from 1 mg to 10 mg |
| Amino acid derivative [from NACA as n- | from 1 mg to 30 mg |
| acetyl-l-cysteine amide] | |
| Enzyme [from papaya proteinase I] | from 5 mg to 50 mg |
| IF [from intrinsic factor] | from 1 mg to 10 mg |
| Bioavailability Enhancerf [from piperine] | from 50 mcg to 500 mcg |

Folate Regulation: The Federal Register Notices from 1971 to 1973 established that increased folate was proper therapy in megaloblastic anemias of tropical and nontropical sprue, nutritional origin, pregnancy, infancy and childhood. Folate metabolism can be affected by malabsorption issues which differ widely among population groups. The Mar. 5, 1996 Federal Register Notice (61 FR 8760) states that “The agency concluded that the scientific literature did not support the superiority of any one source of folate over others, and that the data were insufficient to provide a basis for stating that a specific amount of folate is more effective than another amount [emphasis added].” The actual amount and source of folate require a licensed medical practitioner's supervision to achieve a satisfactory maintenance level, and may exceed the 0.8 mg UL. The Federal Register Notice of Aug. 2, 1973 (38 FR 20750) specifically states that “dietary supplement preparations are available without a prescription (21 CFR 121.1134). Levels higher than dietary supplement amounts are available only with a prescription.

Oral preparations supplying more than 0.8 mg folate per dosage unit would be restricted to prescription dispensing and that a dietary supplement furnishing 0.8 mg could be prescribed when a maintenance level of 0.8 mg per day was indicated . . . . When clinical symptoms have subsided and the blood picture and/or CSF folate levels have become normal, a maintenance level should be used. Patients should be kept under close supervision and adjustment of the maintenance level made if relapse appears imminent. In the presence of alcoholism, hemolytic anemia, anticonvulsant therapy, or chronic infection, the maintenance level may need to be increased.” However, once the level of active folate exceeds 0.8 mg—as prescribed dosages, then the product is no longer a medical food but a prescription (RJ folate I dietary supplement regardless of pregnancy/lactation status even though folic acid—including reduced forms, may be added to medical foods as defined in section S(b)(3) of the Orphan Drug Act (21 USC 360ee(b)(3)), or to food (21 CFR 172.345)11 In the Letter Rg Dietary Supplement Health Claim for Folic Acid, Vitamin B₆, and Vitamin B₁₂ and Vascular Disease (Docket No. 99P-3029) dated Nov. 28, 2000, FDA wrote“ . . . high intakes of folate may partially and temporarily correct pernicious anemia while the neurological damage of vitamin B₁₂ deficiency progresses. IOM/NAS (1998) set the UL for all adults of 1 mg per day because of devastating and irreversible neurological consequences of vitamin B₁₂ deficiency, the data suggesting that pernicious anemia may develop at a younger age in some racial or ethnic groups, and the uncertainty about the extent of the occurrence of Vitamin B₁₂ deficiency in younger age groups.

It should be emphasized that the embodiments described herein are merely possible examples of implementations, merely set forth for a clear understanding of the principles of the present disclosure. Many variations and modifications may be made to the described embodiment(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

One should note that conditional language, such as, among others, “can,” “could,” “might,” or “may,” unless specifically stated otherwise, or otherwise understood within the context as used, is generally intended to convey that certain embodiments include, while alternative embodiments do not include, certain features, elements and/or steps. Thus, such conditional language is not generally intended to imply that features, elements and/or steps are in any way required for one or more particular embodiments or that one or more particular embodiments necessarily include logic for deciding, with or without user input or prompting, whether these features, elements and/or steps are included or are to be performed in any particular embodiment. Unless stated otherwise, it should not be assumed that multiple features, embodiments, solutions, or elements address the same or related problems or needs.

Various implementations described in the present disclosure may include additional systems, methods, features, and advantages, which may not necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

1. A nutritional supplement comprising:
folinic acid, folic acid, L-methylfolate, Vitamin B₁₂, magnesium, N-acetyl-L-cysteine amide, docosahexaenoic acid, phosphatidylcholine-docosahexaenoic acid, and phosphatidylethanolamine-docosahexaenoic acid,
wherein the total amount of combined folinic acid, folic acid, and L-methylfolate is more than 1 mg, and
wherein the total amount of combined phosphatidylcholine-docosahexaenoic acid and phosphatidylethanolamine-docosahexaenoic acid is more than 5 mg.

2. The composition of claim 1 further comprising vitamin D₃, vitamin B₁, vitamin B₂, Coenzyme A, vitamin B₃, vitamin C, and threonate.

3. The composition of claim 1, wherein the folic acid is in the form of a controlled release folic acid.

4. The composition of claim 1, further comprising N-acetyl-L-cysteine, or enzyme from papaya proteinase I, or intrinsic factor or piperine.

5. The composition of claim 1, wherein the composition is a nutritional supplement.

6. A composition consisting essentially of:
folinic acid, folic acid, methylfolic acid, cobalamin, magnesium:N-acetyl-L-cysteine chelate, docosahexaenoic acid, phosphatidylcholine-docosahexaenoic acid, phosphatidylethanolamine-docosahexaenoic acid, Vitamin D₃, Vitamin B₁, Vitamin B₂, Vitamin B₅, Vitamin B₃, Vitamin C, threonate, N-acetyl-L-cysteine amide, papaya proteinase I, intrinsic factor, and piperine,
wherein the total amount of folinic acid, folic acid and L-methylfolate is more than 1 mg, and
wherein the total amount of phosphatidylcholine-docosahexaenoic acid, and phosphatidylethanolamine-docosahexaenoic acid is more than 5 mg.

7. The composition of claim 6, wherein the folinic acid is in the amount of 4 mg, folic acid is in the amount of 0.3 mg, L-methylfolate is in the amount of 0.7 mg, cobalamin is in the amount of 2 mg, magnesium:N-acetyl-L-cysteine chelate is in the amount of 5 mg, docosahexaenoic acid is in the amount of 80 mg, phosphatidylcholine-docosahexaenoic acid is in the amount of 700 mcg, phosphatidylethanolamine-docosahexaenoic acid is in the amount of 300 mcg, vitamin D₃ is in the amount of 100 IU, vitamin B₁ is in the amount of 1 mg, vitamin B₂ is in the amount of 1 mg, Vitamin B₅ is in the amount of 5 mg, vitamin B₃ is in the amount of 1 mg, vitamin C is in the amount of 25 mg, and threonate is in the amount of 5 mg, N-acetyl-L-cysteine amide is in the amount of 10 mg, papaya proteinase I is in the amount of 20 mg, intrinsic factor is in the amount of 5 mg, and piperine is in the amount of 250 mcg.

8. A method of treating cognitive impairment, attention deficit disorder, depression, memory loss, apathy, Alzheimer's Disease, or oxidative stress, the method comprising administering to a patient a composition comprising a therapeutic amount of folinic acid, folic acid, L-methylfolate, cobalamin, magnesium, N-acetyl-L-cysteine, DHA, PC-DHAe, and PE-DHAe.

9. The method of claim 8, wherein the composition further comprises vitamin D₃, vitamin B₁, vitamin B₂, coenzyme A, vitamin B₃, vitamin C, and threonate.

10. The method of claim 8, wherein the folic acid is in the form of a controlled release folic acid.

11. The method of claim 9, wherein the composition further comprises N-acetyl-L-cysteine amide, or papaya proteinase I, or intrinsic factor, or piperine.

12. The method of claim 8, wherein the composition is a nutritional supplement.
